SEQUENCE LISTING (14 sequences)

SEQ ID NO: 1 - DNA, 56 bp, Artificial Sequence
  inventor Uematsu, Chihiro ; Gouda Chifumi; Anazawa Takashi; Shirai Masataka
  Description of Artificial Sequence model target oligo
gtaccacaaggcctttcgcgacccaacactactcggctagcagtctcgcgggggca

SEQ ID NO: 2 - DNA, 99 bp, Artificial Sequence
  Description of Artificial Sequence padlock probe for Hepatitis C Virus
tgttgggtcgcgaaaggccccttctcactgttctctcatagaagtgatgagggaacagagcactcatctcttctccctgttagactgctagccgagtag

SEQ ID NO: 3 - DNA, 21 bp, Artificial Sequence
  Description of Artificial Sequence DNA primer which is used in RCR
ctctgttccctcatcacttct

SEQ ID NO: 4 - DNA, 33 bp, Artificial Sequence
  Description of Artificial Sequence molecular beacon which detects RCR products
cgacgtcccttctcactgttctctcatacgtcg

SEQ ID NO: 5 - DNA, 20 bp, Artificial Sequence
  Description of Artificial Sequence reverse transcription DNA primer which is used in reverse transcription and hybridizes with Hepatitis C Virus RNA
tgctcatggtgcacggtcta

SEQ ID NO: 6 - DNA, 99 bp, Artificial Sequence
  Description of Artificial Sequence padlock probe for Hepatitis B Virus
acytgtattcccatcccacccttctctctcatcactgttagaagtgatgagggaacagagcactcatctcttctccctgttagaaggagaggaaaytgc

SEQ ID NO: 7 - DNA, 21 bp, Artificial Sequence
  Description of Artificial Sequence fluorescent probe which detects RCR products which detects HBV RCR products
cccttctctctcatcactgtt

SEQ ID NO: 8 - DNA, 21 bp, Artificial Sequence
  Description of Artificial Sequence quencher probe for fluorescent probe which detects HBV RCR products
aacagtgatgagagagaaggg

SEQ ID NO: 9 - DNA, 21 bp, Artificial Sequence
  Description of Artificial Sequence quencher probe for fluorescent probe which detects HBV RCR products
aacagtgatgagcgagaaggg

SEQ ID NO: 10 - DNA, 18 bp, Artificial Sequence
  Description of Artificial Sequence quencher probe for fluorescent probe which detects HBV RCR products
agtgatgagagagaaggg

SEQ ID NO: 11 - DNA, 21 bp, Artificial Sequence
  Description of Artificial Sequence fluorescent probe which detects HCV RCR products
cccttctcactgttctctcat

SEQ ID NO: 12 - DNA, 21 bp, Artificial Sequence
  Description of Artificial Sequence DNA oligonucleotide which is used for elimination of secondary structure of RCR products
cactcatctcttctccctgtt

SEQ ID NO: 13 - DNA, 18 bp, Artificial Sequence
  Description of Artificial Sequence quencher probe for fluorescent probe which detects HCV RCR products
agagaacagtgagaaggg

SEQ ID NO: 14 - DNA, 33 bp, Artificial Sequence
  Description of Artificial Sequence molecular beacon which detects RCR products
cgacgtcccttctctctcatcactgttacgtcg

(19) United States
(12) Patent Application Publication
(10) Pub. No.: US 2007/0231808 A1
(43) Pub. Date: Oct. 4, 2007

(54) Methods of nucleic acid analysis by single molecule detection

(75) Inventors: Chifumi Gouda, Kokubunji (JP); Chihiro Uematsu, Kawasaki (JP); Takashi Anazawa, Koganei (JP); Masataka Shirai, Higashimurayama (JP)

Correspondence Address: Stanley P. Fisher;Reed Smith LLP, 3110 Fairview Park Drive, Suite 1400, Falls Church, VA, 22042-4503, US

(73) Assignee: Hitachi High-Technologies Corporation

(21) Appl. No.: 11/441,193

(22) Filed: May 26, 2006

(30) Foreign Application Priority Data
May 31, 2005 (JP) 2005-159878

(51) Int. Cl. C12Q 1/68

(52) U.S. Cl. 435/6

(57) ABSTRACT

This invention provides a method of nucleic acid analysis that enables highly accurate and sensitive quantitation by counting the number of molecules among a plurality of types of genes without amplifying specific genes and that enable reduction of quantitation limits. This method comprises steps of: allowing a polynucleotide comprising a first region having a sequence complementary to the target gene at the 3′ end, a second region having a sequence complementary to the target gene at the 5′ end, and a third region corresponding to a detection probe to hybridize to the target gene; allowing the 3′ end of the first region hybridized to the target gene to ligate to the 5′ end of the second region so as to obtain a circularized polynucleotide; with the use of the circularized polynucleotide as a template, performing a primer extension reaction using a primer having a sequence complementary to part of the circularized polynucleotide and a strand-displacement DNA polymerase; allowing a detection probe containing a sequence identical to the third region to hybridize to a sequence complementary to the third region that iteratively appears in a single-stranded portion of the extension product; and optically detecting the quantity of the detection probe hybridized to the extension product to thereby quantitate the target gene.

### CLAIM OF PRIORITY

The present application claims priority from Japanese application JP 2005-159878 filed on May 31, 2005, the content of which is hereby incorporated by reference into this application.

### TECHNICAL FIELD

The present invention relates to a method of nucleic acid analysis by single molecule detection and a kit used therefor. More particularly, the present invention relates to a method of nucleic acid analysis by single molecule detection, wherein rolling-circle replication (RCR) products are generated in accordance with the number of molecules of the target genes (target nucleic acids), fluorescent probes are allowed to bind specifically to the generated RCR products, and the number of molecules is counted via optical detection, and a kit used therefor.

### BACKGROUND ART

Analysis of the expression levels of specific genes with high sensitivity and high accuracy over a wide dynamic range plays a very important role in the functional analysis of genes, research on or diagnosis of diseases, and the like. For example, infection diagnosis requires the quantitation of infector viral genes at the initial stage in order to avoid expansion of an infection or to effectively treat diseases. In the field of drug manufacturing, it is necessary that the target of drug discovery be identified or the level of gene expression that varies in a disease-specific manner be quantitated in order to evaluate the drug efficacy of a novel drug.

Real-time polymerase chain reaction (PCR) (Klein et al., Electrophoresis, 1999, 20, 291-299) is known as a method for inspecting the level of gene expression. Real-time PCR is carried out by subjecting a standard sample and the target genes existing in a specimen to amplification by PCR in separate reaction vessels and comparing the amplification efficiencies in order to indirectly quantitate the target genes. Since influences such as amplification inhibition may be imposed depending on the sequence of the target gene and the sequence of a contaminating gene contained in a sample, amplification efficiencies vary each time and the accuracy of quantitation may be lowered.

Accuracy of quantitation is varied during the process of amplification. Thus, single molecule detection, which does not require the amplification procedure, is effective for highly accurate quantitation. Single molecule detection involves labeling of target genes in a specimen-containing solution with a fluorophore to visualize them via laser irradiation, and direct assay of the fluorescent-labeled target genes while discriminating such genes from one another. The determination of the number of molecules counted in the volume subject to detection without the amplification of the target genes enables highly accurate quantitation. Examples of methods of discriminating among fluorescent-labeled target genes include those involving the application of evanescent wave incidence (Funatsu et al., Nature, 1995, 374, 555-559) or confocal incidence (JP Patent Publication (Kohyo) No. 2002-528714 A).

Evanescent wave incidence is a laser irradiation technique that is capable of single molecule detection via labeling of a molecule with a single fluorophore under ideal conditions. In this technique, a molecule is selectively irradiated with a laser to a depth of about 50 nm from the cell surface. If an image of a 50 μm×50 μm field is obtained every second, the volume subject to detection becomes 7.5×10⁻¹¹ l in 10 minutes. In principle, single molecule detection can be carried out if a single molecule is present in the volume subject to detection. Thus, the quantitation limits are 10 ⁻¹⁴ mol/l. In the case of confocal incidence, the volume subject to detection is 1.0×10⁻¹⁴ l in 10 minutes and the quantitation limits are 10⁻⁹ mol/l according to a similar calculation. In contrast, 10 μl of specimens is detected in 2 hours of amplification via real-time PCR. Since amplification can be carried out if a specimen contains 10 molecules, the quantitation limits are 10⁻¹⁸ mol/l. The quantitation limits obtained via evanescent wave incidence or confocal incidence are higher than those of real-time PCR by 4 or 8 orders of magnitude because of the small volume subject to detection. Accordingly, it is necessary that the volume subject to detection be increased and the quantitation limits be rendered equivalent to those of real-time PCR in order to realize highly accurate gene quantitation by single molecule detection.

As a laser irradiation technique that can increase the volume subject to detection by single molecule detection, lateral incidence of a sheet-like laser beam has been reported (Anazawa et al., Anal. Chem, 2002, 74, 5033-5038). With this technique that involves the application of a 20-μm-thick laser, 3.0×10⁻⁸ l of specimen-containing solution can be assayed in an amount that is approximately 400 times larger than the amount assayed via evanescent wave incidence by obtaining an image of a 50 μm×50 μm field every second and performing detection for 10 minutes. Via a further expansion of the field to 250 μm×250 μm to increase the volume subject to detection, the quantitation can be carried out to quantitation limits equivalent to those of real-time PCR. In comparison with evanescent wave incidence, however, the lateral incidence of a sheet-like laser beam suffers from deteriorated sensitivity in terms of single molecule detection because of a lowered laser intensity or an increased background intensity due to an increased volume subject to detection in the thickness direction. In order to perform single molecule detection with lateral incidence, therefore, it is necessary that a single molecule of the target gene be labeled specifically with a plurality of fluorophores to render the molecule detectable. In the aforementioned report, YOYO is employed as a fluorophore. Since YOYO is a fluorophore that nonspecifically binds to DNA in amounts of 1 YOYO for every 5 bases, it is impossible to specifically label a specific gene. In order to impart specificity, a molecular beacon or fluorescent probe is allowed to hybridize to the target gene to specifically label such target gene.

In order to perform more accurate quantitation, it is preferable that fluorophores that have bound to the target gene selectively emit fluorescence but that free fluorophores do not emit fluorescence.

Rolling circle replication (RCR) is known as a method of labeling a single molecule with a plurality of identical fluorophores (U.S. Pat. No. 5,854,033). An RCR product obtained via such technique comprises a repeat of probe binding sites, and thus, a single molecule of the RCR product is labeled with a plurality of fluorophores. The RCR reaction produces the target genes and the RCR products at a ratio of 1:1. Accordingly, the RCR products can be subjected to single molecule detection to count the molecules, which enables quantitation of the target genes. As an application of the RCR technique, for example, Lizardi et al. performed SNP detection via the solid-phase RCR reaction, labeled the RCR products derived from the wild type and from the mutant type with fluorescent probes derived from different fluorophores, washed the labeled products, performed single molecule detection, and determined abundance via molecule counting to perform relative quantitation (Lizardi et al., Nature Genetics, 1998, 19, 225-232). Blab et al. have labeled the RCR products with fluorescent probes and have conducted homogeneous single molecule detection in a solution containing enormously excessive amounts of free fluorescent probes (Blab et al., Anal. Chem, 2004, 76, 495-498). In this technique, however, excess targets are mixed with the padlock probes, and thus the targets cannot be quantitated.

### DISCLOSURE OF THE INVENTION

As described above, conventional techniques have been insufficient in terms of performing highly accurate and highly sensitive gene quantitation (nucleic acid quantitation). Accordingly, objects of the present invention are to overcome such drawbacks of conventional techniques, to perform highly accurate and sensitive quantitation by counting the number of molecules among a plurality of types of genes without amplifying specific genes, and to reduce quantitation limits.

In a conventional technique of single molecule detection whereby a target molecule labeled with a single fluorophore is detected, the volume subject to detection was small, and the quantitation limits were high. Thus, such a technique could not be applied to actual quantitation. If a volume subject to detection is increased by single molecule detection by lateral incidence to overcome such a drawback, the detection could not be realized via simple labeling of the target molecule with a single fluorophore because of a decreased laser density, an increased background, or the like. That is, it is necessary that a single target molecule be labeled with a plurality of fluorophores to increase the fluorescence intensity per target molecule. In the present invention, therefore, a single molecule was labeled with a plurality of identical fluorophores via RCR, which enabled the provision of fluorescence intensity that could be applied to single molecule detection with lateral incidence.

Specifically, the present invention provides a method of gene quantitation comprising steps of:

allowing a polynucleotide comprising a first region having a sequence complementary to the target gene at the 3′ end, a second region having a sequence complementary to the target gene at the 5′ end, and a third region corresponding to a detection probe to hybridize to the target gene;

allowing the 3′ end of the first region hybridized to the target gene to ligate to the 5′ end of the second region so as to obtain a circularized polynucleotide;

with the use of the circularized polynucleotide as a template, performing a primer extension reaction using a primer having a sequence complementary to part of the circularized polynucleotide and a strand-displacement DNA polymerase;

allowing a detection probe containing a sequence identical to the third region to hybridize to a sequence complementary to the third region that iteratively appears in a single-stranded portion of the extension product; and

optically quantitating the amount of the detection probe hybridized to the reaction product.

As described below, the detection probe may be a normal molecular beacon, and a fluorescent probe may be used together with a quencher probe corresponding thereto. Further, an oligomer comprising a sequence identical to an arbitrary region on the aforementioned polynucleotide (a fourth region that does not overlap with the third region) may be used for resolving a higher-order structure of the extension product.

In the present invention, specifically, excess padlock probes are allowed to act on the target genes in a given known volume of solution to generate circulated padlock probes corresponding to the target genes. The circulated padlock probes are subjected to hybridization with primers so as to perform RCR reactions, and the resulting RCR products are specifically fluorescent-labeled. In order to prevent molecular loss and to maintain quantitation accuracy, single molecule detection is carried out in a homogeneous system without the performance of BF separation, and the number of molecules is counted by identifying an individual molecule of the RCR product that has been fluorescent-labeled. Detection is carried out with lateral incidence, the volume subject to detection over 10 minutes is set to at least approximately 3.0×10⁻⁸ l, and quantitation limits of approximately 10⁻¹⁸ mol/l are realized. Since the RCR product is generated in relation to the target genes, determination of the number of molecules of the RCR product leads to determination of the number of molecules of the target genes. This enables quantitation of the target genes. Thus, highly accurate quantitation can be realized by counting the number of molecules among a plurality of types of genes without amplifying specific genes.

According to the method of the present invention, a padlock probe that recognizes a target gene (the target nucleic acid) comprises: a first base sequence portion that recognizes a base sequence of the target gene at the 5′ end; a second base sequence portion that recognizes a base sequence of the target gene at the 3′ end, and a portion to which a primer hybridizes. Such padlock probe further comprises a third base sequence portion that is identical to a molecular beacon. In the presence of the target gene, the padlock probe recognizes the sequence of the target gene and hybridizes thereto. Thereafter, the padlock probe is circularized via ligation and it becomes a circularized padlock probe. A primer hybridizes to this circularized padlock probe and extends, and the RCR reaction proceeds. The RCR product is a single-stranded nucleic acid that is generated in the following manner. A circularized padlock probe is used as a template, and a primer is continuously extended on the circularized template prove by displacing a strand that have been synthesized (see FIG. 1.) Thus, the RCR product contains a repeat of a base sequence complementary to the third base sequence. Upon hybridization of a molecular beacon having a base sequence identical to the third base sequence to the RCR product (labeled at the 5′ end with a fluorophore), the RCR product is labeled with a plurality of fluorophores.

The aforementioned padlock probe can be arbitrarily designed, except for the portion of the sequence that recognizes the target gene. The third base sequence contained in the padlock probe, i.e., the base sequence identical to the molecular beacon, can be arbitrarily designed regardless of the target gene sequence. Also, a primer sequence can be arbitrarily designed regardless of the target gene sequence. In the padlock probe, a sequence to which a primer hybridizes and the third base sequence may be continuously present, or a joining segment may be present between such sequences. Based on such configurations and functions, the padlock probe is preferably composed of approximately 60 to 200 bases.

As a probe that labels an RCR product, a fluorescent probe having the third base sequence and a quencher probe having a base sequence complementary to part of the third base sequence may be used instead of the molecular beacon having the third base sequence. Since the fluorescent probe is labeled with a fluorophore at its 3′ end, the RCR product becomes labeled with a plurality of fluorescent probes upon hybridization of the RCR product to the fluorescent probes. After the labeling of the RCR product, a quencher probe having a base sequence complementary to part of the fluorescent probe, i.e., a base sequence complementary to part of the third base sequence, is allowed to hybridize to a fluorescent probe that has not hybridized to the RCR product. Since the quencher probe is labeled with a quencher at its 5′ end, the quencher comes into contact with a fluorophore located at the 3′ end of the fluorescent probe that has hybridized to the quencher probe, and fluorescence is quenched. As a result, fluorescence derived from a fluorescent probe that has not hybridized to the RCR product is quenched, and a fluorescent probe that has hybridized to the RCR product is selectively allowed to emit fluorescence. This enables homogeneous single molecule detection while suppressing the background and quantitation of the target genes.

In the above method, a quencher probe having a base sequence complementary to part of the third base sequence is added to a reaction solution in order to quench excess fluorescent probes existing therein. The base sequence of the quencher probe may not be completely complementary to the third base sequence, and it may contain a base-pair mismatch in a part thereof. Thermostability in hybridization between the fluorescent probe containing a base-pair mismatch and the quencher probe becomes lower than the thermostability attained with the use of a fluorescent probe containing no base-pair mismatch. Accordingly, the quencher probe does not bind to the fluorescent probe that has hybridized to the RCR product, it selectively hybridizes to excess free fluorescent probes existing in the reaction solution, and it preferentially quenches fluorescence derived from fluorescent probes that are not involved in the labeling of the RCR product. One or two such base-pair mismatches can be introduced into the quencher probe.

Similar effects may be attained by differentiating the hybridization thermostability by shortening the base sequence of the quencher probe by 1 to 5 bases to prevent the quencher probes from peeling the fluorescent probes that had hybridized to the RCR product.

In order to assuredly quench free fluorescent probes, quencher probes are preferably added after the initiation of hybridization between the fluorescent probes and the RCR products. Also, it is preferable that the quantity of the quencher probes added be greater than that of the fluorescent probes, such as twice or more the quantity of the fluorescent probes in terms of the molar ratio.

The 3′ end of a fluorescent probe or molecular beacon having the third base sequence is labeled with a fluorophore or quencher, phosphorylated, or dideoxidized to prevent extension after the hybridization. Also, the 3′ end of the quencher probe having a base sequence complementary to part of the third base sequence is labeled with a quencher, phosphorylated, or dideoxidized to prevent extension after the hybridization with a fluorescent probe having the third base sequence.

The RCR product generated by the method of the present invention is a single-stranded nucleic acid. Thus, it may form a higher-order structure upon an intramolecular hydrogen bond. An oligonucleotide having a sequence identical to a fourth base sequence contained in a padlock probe may be used as a resolution oligomer of higher-order structure. In the presence of the resolution oligomer of higher-order structure, the RCR product hybridizes to the resolution oligomer of higher-order structure. Thus, the higher-order structure of the RCR product can be resolved.

The resolution oligomer of higher-order structure having a sequence identical to the fourth base sequence is designed to hybridize to the RCR product. In order to avoid the extension after hybridization, the 3′ end is phosphorylated or dideoxidized. This can prevent extension of the fluorescent probe, the quencher probe, and the resolution oligomer of higher-order structure that are used in the present invention, and thus, they would not inhibit the generation of the RCR product.

The molecular beacon, the fluorescent probe, the quencher probe, and the resolution oligomer of higher-order structure that are used in the present invention are each preferably composed of approximately 15 to 30 bases.

When a plurality of molecular beacons (or fluorescent probes and quencher probes) corresponding to a plurality of genes are used in simultaneous analysis of such plurality of genes, module-shuffling sequences (JP Patent Publication (Kokai) No. 2000-106900 A) may be employed in order to prevent cross-hybridization between molecular beacons (or between fluorescent probes and quencher probes). When a plurality of primers or resolution oligomers of higher-order structure corresponding to a plurality of genes are used, module-shuffling sequences may also be employed for the same reasons.

The present invention also provides a probe kit for assaying fluorescent intensity used for single molecule detection. The kit according to the present invention is essentially composed of a padlock probe that recognizes the target gene, a molecular beacon having a sequence identical to the third base sequence contained in the padlock probe, and a primer for generating an RCR product.

According to the present invention, a single molecule can be labeled with a plurality of fluorophores via an RCR reaction. While the volume to be irradiated with a laser is expanded with lateral incidence to result in a large volume subject to detection, single molecule detection can be carried out with sufficiently high sensitivity. This enables the quantitation of the target genes by single molecule detection and accurate quantitation of the target genes at quantitation limits equivalent to those for conventional real-time PCR.

### BRIEF DESCRIPTION OF THE DRAWINGS

FIG. 1 shows a procedure for fluorescent labeling an RCR product using a molecular beacon, performing single molecule detection, counting the number of molecules in the RCR product, and quantitating the target genes.

FIG. 2 shows a procedure for fluorescent labeling an RCR product using a fluorescent probe, lowering background fluorescence using a quencher probe, performing single molecule detection, counting the number of molecules of the RCR product, and quantitating the target genes.

FIG. 3 schematically shows the correlation of two strands upon hybridization between a fluorescent probe and a quencher probe.

FIG. 4 shows a procedure for improving the labeling efficiency of fluorescent labeling of the RCR product with the use of a resolution oligomer of higher-order structure as shown in FIG. 2, performing single molecule detection, counting the number of molecules of the RCR product, and quantitating the target genes.

FIG. 5 schematically shows the structure of an apparatus for single molecule detection used for detecting a fluorescent-labeled RCR product.

FIG. 6 schematically shows the structure of a padlock probe used for simultaneous detection of a plurality of target genes.

FIG. 7 schematically shows the structure of a detection section of an apparatus that discriminates and detects a plurality of fluorescent wavelengths.

FIG. 8 shows the results of counting the number of molecules of the RCR products generated from target oligos at various concentrations by single molecule detection.

FIG. 9 is a chart showing the results of hepatitis C virus quantitation.

FIG. 10 shows images of ROX-labeled RCR products assayed with the use of various types of quencher probes.

FIG. 11 shows images showing the results of assaying FAM-labeled RCR products.

FIG. 12 is a chart showing the results of simultaneously quantitating two types of viruses.

### PREFERRED EMBODIMENTS OF THE INVENTION

Hereafter, the present invention is described in detail with reference to the drawings.

1. Design of Padlock Probe and Molecular Beacon

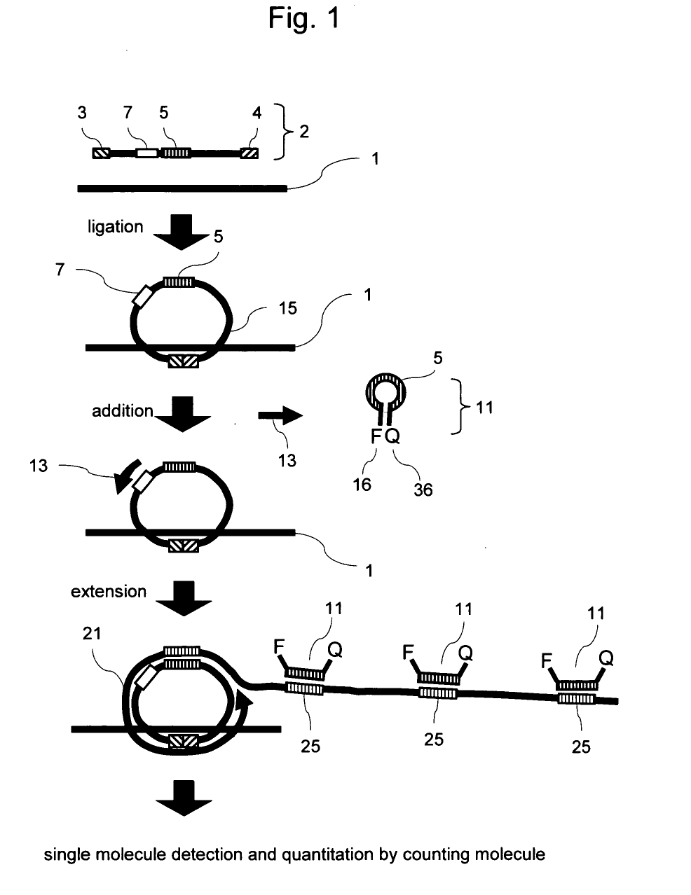

FIG. 1 schematically shows fluorescent labeling of the RCR product of the present invention, wherein the numerical reference 1 indicates a target gene and the numerical reference 2 indicates a padlock probe that recognizes the target gene. The padlock probe 2 comprises a sequence portion 3 that hybridizes to the target gene at its 5′ end, a sequence portion 4 that hybridizes to the target gene at its 3′ end, a sequence portion 5 that consists of a sequence identical to the sequence in the molecular beacon 11 used for detection, and a sequence portion 7 that hybridizes to a primer 13. The sequence portion 3 and the sequence portion 4 are designed to be located at the 5′ end and at the 3′ end of the padlock probe, respectively, and the probe is circularized via ligation to the target gene to which the probe has hybridized.

The sequence portions 3, 4, 5, and 7 may be continuously present on the probe, or joining segments may be present therebetween. The lengths of the sequence portion 3 and of the sequence portion 4 are not particularly limited, and they may be preferably composed of approximately 15 to 30 bases. The length of the sequence portion 5 is not particularly limited, and it is preferably composed of approximately 15 to 30 bases. The length of the sequence portion 7 is not particularly limited, and such sequence is preferably composed of approximately 15 to 30 bases. It is necessary that the sequence portion 3 and the sequence portion 4 be redesigned in accordance with the target genes. However, the sequence portions 5 and 7 can be arbitrarily designed regardless of the target gene sequences, and these portions can be commonly used for all the target genes. Based on such configurations and functions, the padlock probe is preferably composed of approximately 60 to 200 bases.

The molecular beacon 11 is designed to have a sequence identical to the sequence portion 5 of the padlock probe 2, a fluorophore 16 at the 5′ end, and a quencher 36 at the 3′ end. The primer 13 is designed to have a sequence complementary to the sequence portion 7 of the padlock probe 2.

2. Circularization of Padlock Probe

The aforementioned padlock probe recognizes a given sequence on the target gene, hybridizes thereto, and ligates the sequence portion 3 to the sequence portion 4. Thus, the padlock probe is circularized. When the target gene is RNA, it is converted into cDNA in accordance with a technique known in the art. When the target gene to be detected is DNA, it can be used in that state. As shown in FIG. 1, the designed padlock probe 2 is allowed to react with the target gene 1 existing in a given reaction solution, a ligase is added thereto, and the resultant is incubated at 35° C. to 45° C. for approximately 30 to 60 minutes. Thus, the padlock probe is circularized and a circularized padlock probe 15 is obtained.

The circularized padlock probe 15 hybridizes to the target gene 1 at the sequence portion 3 and the sequence portion 4 that recognizes the target gene. The circularized padlock probe 15 comprises a sequence portion 5 consisting of a sequence identical to the sequence in the molecular beacon 11 used for detection and a sequence portion 7 to which the primer 13 hybridizes.

3. RCR Reaction and Labeling

To the circularized padlock probe 15 prepared in the above section, the primer 13 for generating an RCR product and the molecular beacon 11 for detecting the RCR product are added, and an RCR reaction is carried out in a given reaction solution in the presence of strand-displacement DNA polymerase and a substrate. With the aid of strand-displacement DNA polymerase, the 3′ end of the primer 13 is continuously extended on the circularized padlock probe 15 as a template by displacing a strand that have been synthesized. Thus, an RCR product 21 is generated. As the reaction advances, a sequence portion 25 complementary to the sequence portion 5 is iteratively synthesized and contained in the RCR product 21. The molecular beacon 11 hybridizes to the sequence portion 25.

The molecular beacon 11 is labeled with a fluorophore 16 and a quencher 36 indicated as “F” and “Q,” respectively, in FIG. 1. The fluorophore 16 and the quencher 36 may label either the 5′ or 3′ end of the molecular beacon 11. Examples of fluorophores that can be used include coumalin, fluorescein, tetrachlorofluorescein, hexacholorofluorescein, Lucifer yellow, rhodamine, BODIPY, tetramethylrhodamine, Cy3, Cy5, Cy7, eosin, Texas Red, ROX, FAM, and VIC. Examples of quenchers include BHQ1, BHQ2, TAMRA, DABMI, methyl red, and 4-(4′-dimethylaminophenylazo)benzoic acid (DABCYL). A quencher itself is a kind of fluorophore. That is, when fluorophores come into contact with other fluorophores, some fluorophores absorb and quench the fluorescence emitted by the others. Thus, an adequate combination of two entities, whereby fluorescence emitted by one entity (a fluorophore) is absorbed by the other entity (a quencher), is selected from among the aforementioned fluorophores and quenchers and employed as a combination of a fluorophore and a quencher.

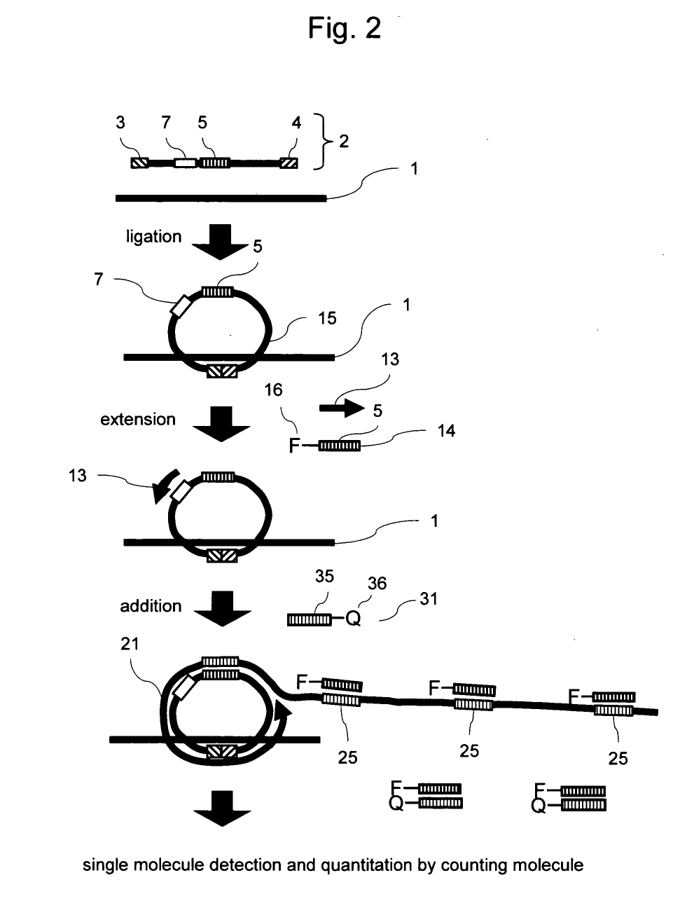

In order to detect an RCR product, labeling may be carried out using a fluorescent probe 14 having a sequence portion 5, as shown in FIG. 2. The fluorescent probe 14 hybridizes to a sequence portion 25 that is iteratively contained in the RCR product. After the fluorescent probe 14 has hybridized to an RCR product 21, a quencher probe 31 is added to the reaction solution. The quencher probe 31 contains part of a sequence portion complementary to the sequence portion 5 of the fluorescent probe 14, i.e., a sequence portion 35, which is part of the sequence portion 25. The quencher probe 31 is labeled with a quencher 36, indicated as “Q” in the figure. A quencher that can be used for the aforementioned molecular beacon can be employed.

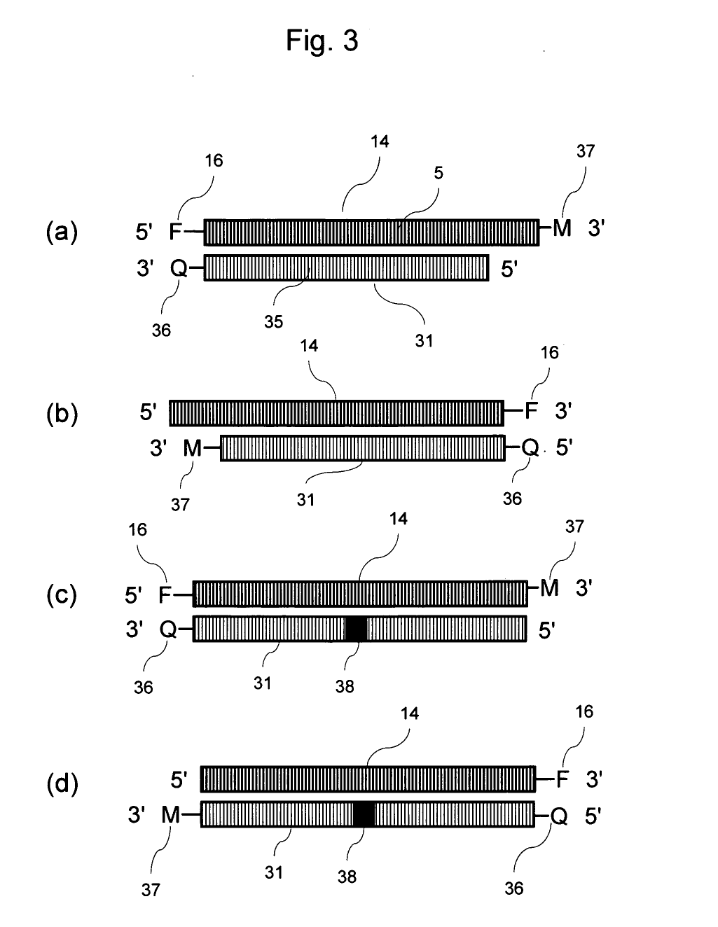

The quencher probe 31 is designed to have a Tm value that is 3° C. to 15° C., and preferably approximately 5° C., lower than that of the fluorescent probe 14, in order to avoid inhibiting the hybridization between the fluorescent probe 14 and the RCR product 21. In order to keep the Tm value low, the length of the quencher probe 31 is set to be 1 to 5 bases shorter than the fluorescent probe 14, as shown in FIG. 3(a) or FIG. 3(b). As shown in FIG. 3(c) or 3(d), a sequence portion 35 that constitutes the quencher probe 31 may not be completely complementary to the sequence portion 5 that constitutes the fluorescent probe 14. A base-pair mismatch may be provided between the quencher probe and the fluorescent probe via introduction of 1 or 2 base-pair mismatches 38 in part of the sequence.

When the 5′ end of the fluorescent probe 14 is labeled with a fluorophore 16 as shown in FIG. 3(a) or 3(c), the 3′ end of the quencher probe 31 is labeled with a quencher 36 so as to bring the fluorophore 16 into close contact with the quencher 36 to thereby quench the fluorescence emitted by the fluorophore 16 by a mechanism of fluorescent energy transfer, upon hybridization between the fluorescent probe 14 and the quencher probe 31. When the 3′ end of the fluorescent probe is labeled with a fluorophore 16 as shown in FIG. 3(b) or 3(d), the 5′ end of the quencher probe 31 is labeled with the quencher 36 so as to bring the fluorophore 16 into close contact with the quencher 36 to thereby quench the fluorescence emitted by the fluorophore 16 by a mechanism of fluorescent energy transfer, upon hybridization between the fluorescent probe 11 and the quencher probe 31.

When the 5′ end of the fluorescent probe 14 is labeled with the fluorophore 16 as shown in FIG. 3(a) and FIG. 3(c), the 3′ end of the fluorescent probe 14 is provided with a terminal modification 37, such as phosphorylation or dideoxydization, to avoid extension induced by DNA polymerase. When the 3′ end of the fluorescent probe 14 is labeled with the fluorophore 16 as shown in FIG. 3(b) and FIG. 3(d), the fluorescent probe 14 will not be extended by DNA polymerase even if the terminal modification as described above is not provided.

Similarly, when the 5′ end of the quencher probe 31 is labeled with the quencher 36 as shown in FIGS. 3(b) and 3(d), it is necessary that the 3′ end of the quencher probe 31 be provided with a terminal modification 37, such as phosphorylation or dideoxydization, to avoid extension induced by DNA polymerase. When the 3′ end of the quencher probe 31 is labeled with the quencher 36 as shown in FIGS. 3(a) and 3(c), the quencher probe 31 will not be extended by DNA polymerase even if the terminal modification is not provided.

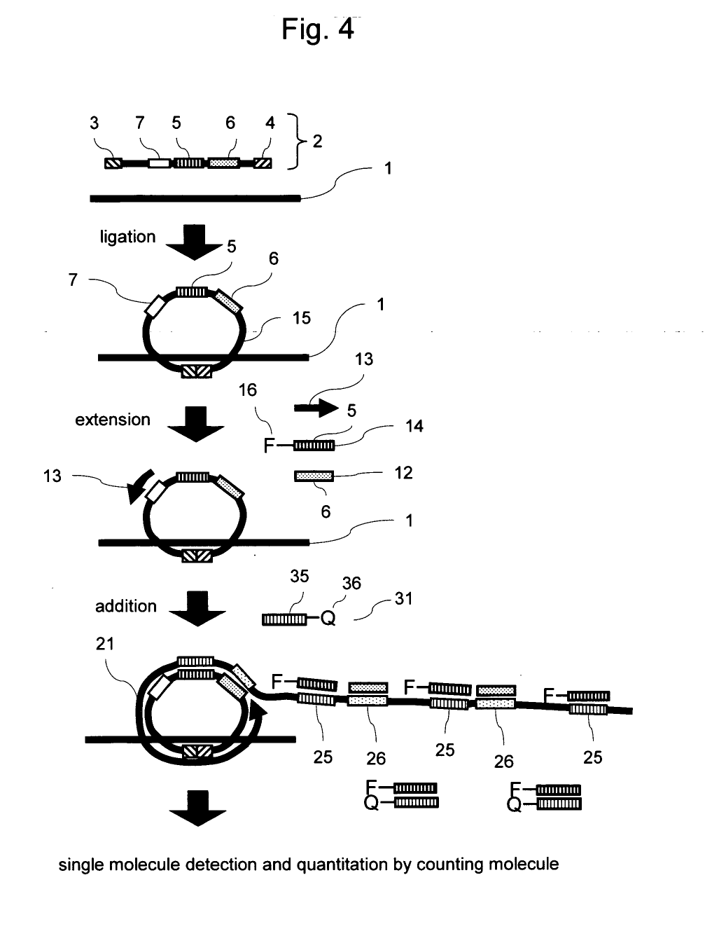

The RCR product 21 is single-stranded DNA, and thus, it may form a higher-order structure in the molecule and cause the efficiency of fluorescent labeling to deteriorate. Such higher-order structure can be resolved with the use of the resolution oligomer of higher-order structure 12 as shown in FIG. 4. The padlock probe 2 is designed to contain a sequence portion 6 consisting of a sequence identical to the resolution oligomer of higher-order structure 12, as shown in FIG. 4. The length of the sequence portion 6 is not particularly limited, and such sequence is preferably composed of approximately 15 to 30 bases. Thus, the RCR product generated from the padlock probe template contains a repeat of sequences 26 complementary to the resolution oligomer of higher-order structure 12.

As shown in FIG. 4, the resolution oligomer of higher-order structure 12 hybridizes to the sequence portion 26 that is iteratively contained in the RCR product 21 to form a plurality of double-stranded structures in the RCR product 21. Consequently, the RCR product 21 becomes incapable of forming the higher-order structure via an intramolecular bond, and the fluorescent probe 14 efficiently hybridizes to the sequence portion 25. The 3′ end of the resolution oligomer of higher-order structure 12 is phosphorylated or dideoxidized in advance to avoid extension by DNA polymerase. The resolution oligomer of higher-order structure 12 can be used in a similar manner when labeling the RCR product with the molecular beacon 11 as shown in FIG. 1.

4. Detection

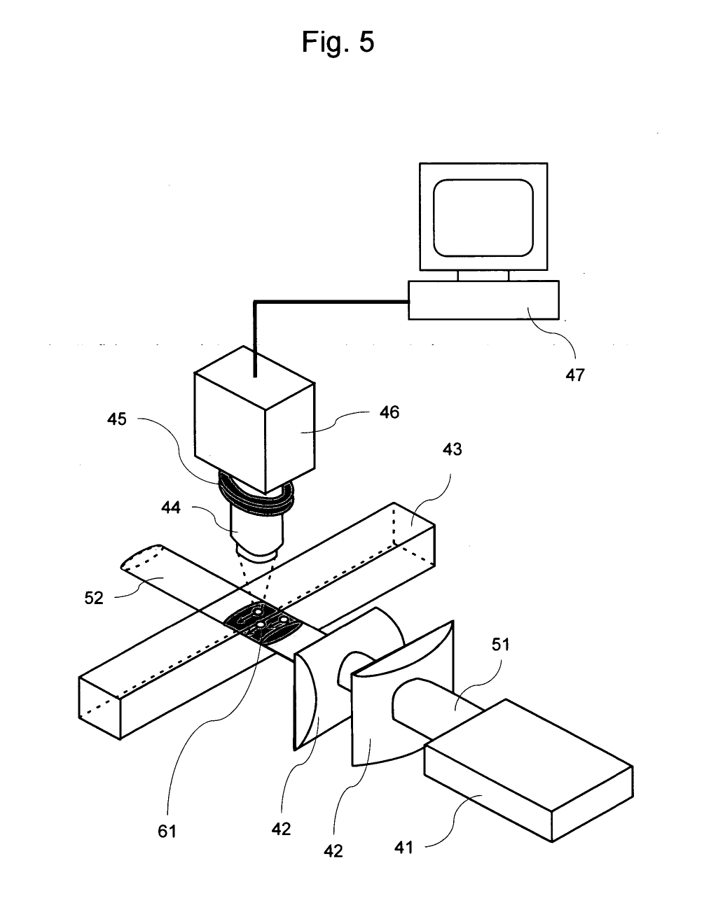

The RCR product is subjected to detection with the use of an apparatus as shown in FIG. 5. A laser beam 51 oscillated from a laser source 41 becomes a sheet-like laser beam 52 through two cylindrical lenses 42. A square capillary 43 is irradiated laterally with a laser beam. A fluorescent-labeled sample 61 in the square capillary 43 is excited by the sheet-like laser beam 52 and emits fluorescence. The fluorescence passes through an objective lens 44 and a filter 45 provided on top of the square capillary 43, and an image is acquired via a charge-coupled device (CCD) camera 46. The acquired image is transferred to a PC 47, recorded, and then analyzed as data. The inner diameter of the square capillary is 50 μm×50 μm, the width of the sheet-like laser beam is 50 μm, and the thickness of the sheet-like laser beam is 20 μm. If an image is acquired with a CCD camera once every second, the volume subject to detection becomes 5×10⁻¹¹ l/sec. The number of molecules in the acquired image is determined, and the density of the target genes in the sample can be determined based on the number of molecules in a given volume subject to detection.

5. Simultaneous Analysis of a Plurality of Target Genes

A method for simultaneously analyzing two or more types of target genes is next described. When analyzing a plurality of target genes simultaneously, it is necessary that a plurality of padlock probes and molecular beacons (or fluorescent probes and quencher probes) be designed.

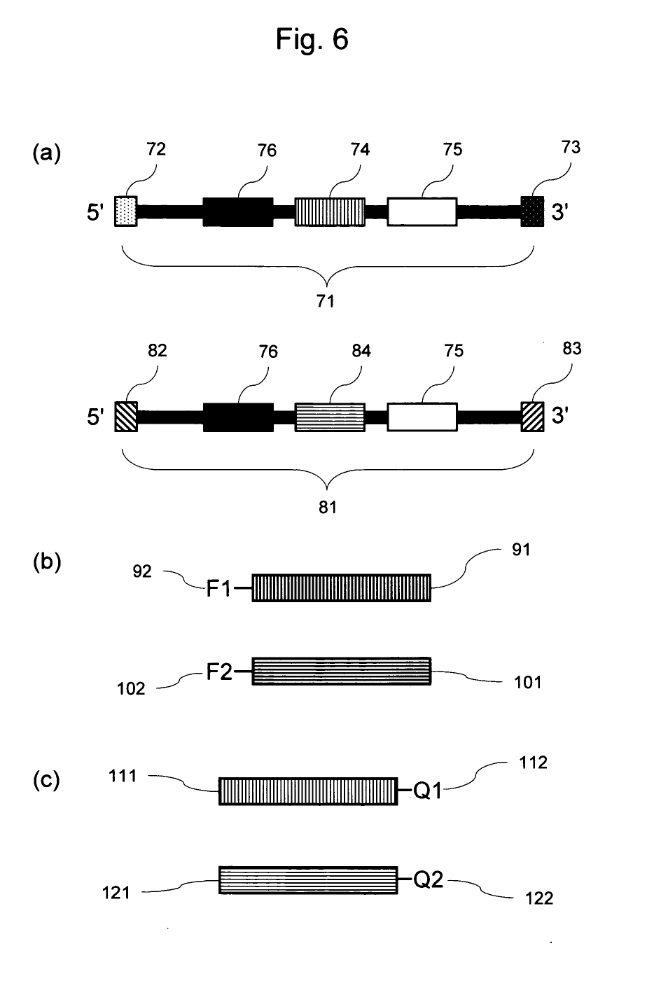

FIG. 6(a) schematically shows the structures of padlock probes that are used for simultaneous analysis of a plurality of target genes. FIG. 6(a) shows padlock probes 71 and 81 that are used for simultaneous analysis of two types of target genes. The sequence portions 72 and 82 of the padlock probes that recognize the target genes at the 5′ ends, the sequence portions 73 and 83 that recognize the target genes at the 3′ ends, and the sequence portions 74 and 84 that are identical to the fluorescent probes are designed to be different sequences. The sequence portion 75 that is identical to the resolution oligomer of higher-order structure may be the same or different from the sequence portion 76 to which a primer hybridizes.

FIG. 6(b) schematically shows the structures of fluorescent probes that are used for simultaneous analysis of a plurality of target genes. The fluorescent probes 91 and 101 consist of the sequences identical to the sequence portions 74 and 84 contained in the padlock probes, and thus a different probe is used for each target gene. The fluorescent probes 91 and 101 are labeled with fluorophores 92 and 102 indicated as F1 and F2 in the figure, respectively.

FIG. 6(c) schematically shows the structures of quencher probes that are used for simultaneous analysis of a plurality of target genes. The quencher probes 111 and 121 consist of sequences complementary to the sequence portions 74 and 84 contained in the padlock probes, and thus a different probe is used for each target gene. The quencher probes 111 and 121 are labeled with quenchers 112 and 122, respectively, that can quench fluorophores 92 and 102 indicated as F1 and F2 in the figure. When the fluorophores 92 and 102 can be quenched with a single type of quencher, use of a single type of quencher may be sufficient. Since the quench efficiency of a quencher varies depending on a type of fluorophore, quenchers Q1 and Q2 may be of the different types when an optimal quencher for the fluorophore F1 differs from that for the fluorophore F2.

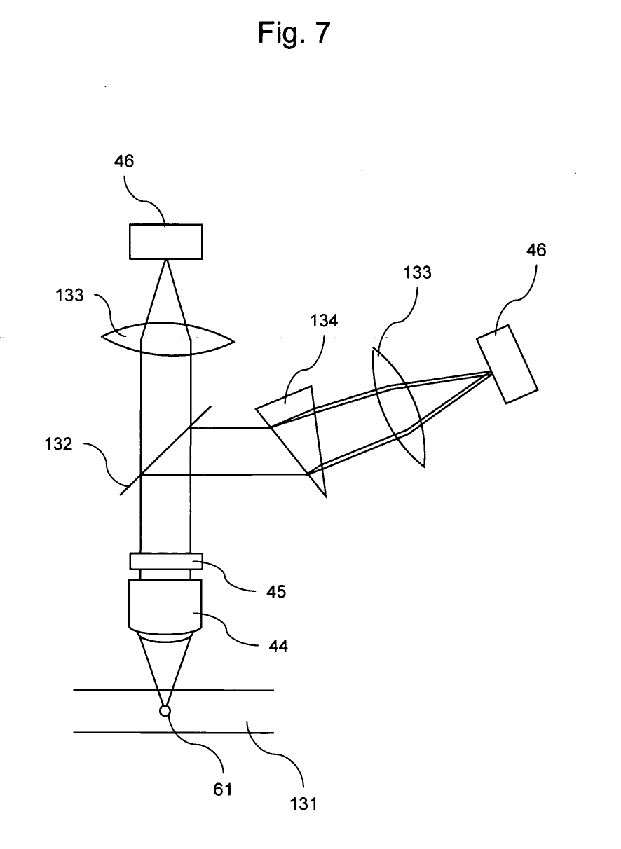

The resolution oligomers of higher-order structure and the primers used for simultaneous analysis of a plurality of target genes may be the same or different. A procedure of analysis is as shown in FIG. 1 and consists of the following steps. A plurality of padlock probes are added to a plurality of types of target genes for circularization, primers are added to perform an RCR reaction, and a plurality of types of RCR products are generated in the same reaction vessel. Thereafter, formation of the higher-order structures of the RCR product is blocked with the use of a plurality of types of resolution oligomers of higher-order structure, the RCR products are labeled with a plurality of types of fluorescent probes, and excess fluorescent probes are quenched with the use of a plurality of types of quencher probes. Detection is carried out through these steps with the use of an apparatus that can discriminate among and detect a plurality of fluorescent wavelengths as shown in FIG. 7.

FIG. 7 shows the structure of a detection section of an apparatus that discriminates among and detects a plurality of fluorescent wavelengths. Fluorescence emitted from the fluorescent-labeled sample 61 passes through an objective lens 44 and a filter 45 provided on top of the capillary 131. It is split into two portions by a beam splitter 132. One of the split fluorescence portions forms an image by a lens 133 without wavelength dispersion, and an image is acquired (an image without wavelength dispersion) via a CCD camera 46. The other fluorescence portion undergoes wavelength dispersion by a prism 134, it forms an image by the other lens 133, and an image is then acquired via another CCD camera 46 (an image with wavelength dispersion). Based on the positional relationship between the image without wavelength dispersion and the image with wavelength dispersion and the distribution of the fluorescent intensities of the image with wavelength dispersion, the fluorescent spectra of the emitted fluorescence are obtained to discriminate among fluorophores. By determining the number of molecules labeled with fluorophores, two or more types of target genes are simultaneously quantitated.

When a plurality of molecular beacons (or fluorescent probes and quencher probes) corresponding to a plurality of genes are used for simultaneous analysis of such plurality of genes, module-shuffling sequences (JP Patent Publication (Kokai) No. 2000-106900 A) may be employed in order to prevent cross-hybridization between molecular beacons (or between fluorescent probes and quencher probes). When a plurality of primers or resolution oligomers of higher-order structure corresponding to a plurality of genes are used, module-shuffling sequences may also be employed for the same reasons.

The term “module-shuffling sequences” refers to a plurality of sequences prepared by rearranging the order of a plurality of module sequences having the same bases at the both ends and composed of 3 or 4 bases. Oligonucleotides (or polynucleotides) having module-shuffling sequences hybridize to the target genes of interest with the same kinetics. For example, it is assumed that sequences of two probes, i.e., probe A and probe B, corresponding to the gene A and the gene B are composed of module sequences comprising 3 or 4 bases. The number of module sequences constituting probes A and B is not particularly limited, and it is generally approximately 5 to 8. The bases at both ends of the module sequences are composed of the same base type. The probe A sequence is composed by rearranging the order of module sequences having the same bases at both ends with probe B sequence. Since the order of the module sequences having bases of the same type at both ends is rearranged, the base sequences at the joining segments of module sequences are identical to each other with regard to probe A and probe B. Modules constituting the probe sequences are the same in probe A and probe B. This renders the thermodynamic properties of probe A equivalent to those of probe B, and their Tm values are identical to each other according to the nearest neighbor method. Although the entire sequence of probe A is different from that of probe B, they have substantially the same Tm values, and they can hybridize to their complementary sequences with the same kinetics even if they are allowed to simultaneously react in the same reaction tube. When these probes are employed for quantitative analysis, cross-hybridization can be prevented, and more accurate analysis can be performed. In the foregoing description, the designing of two types of probes was described. Three or more types of probes can also be designed in the same manner.

6. Kit

As described above, a padlock probe that recognizes the target gene sequence is used, the padlock probe is circularized in accordance with the presence or absence of the target gene, a primer is allowed to hybridize to the circularized padlock probe to perform an RCR reaction, the generated RCR product is labeled with a molecular beacon, and the labeled product is subjected to single molecule detection so as to count the number of molecules of the RCR product. Thus, the target genes can be quantitated. When the other target gene is to be detected, the sequence of the padlock probe that recognizes the target gene may be selectively modified. Since the type of primer or molecular beacon is independent of the base sequence of the target gene, a universal primer or molecular beacon can be employed, regardless of the target gene.

Specifically, the present invention provides a kit for nucleic acid quantitation comprising a primer and a probe that can be universally employed. A kit may be for analyzing a single type or a plurality of types of target genes. The characteristics and the structures of the padlock probes, the primers, and the molecular beacons, which are essential constituents of the kit, are as described above.

The kit according to the present invention may comprise other reagents and the like that are necessary for quantitative gene analysis, in addition to the essential constituents, i.e., the padlock probes, the primers, and the molecular beacons. Examples thereof include enzymes (strand-displacement DNA polymerase), buffers that realize preferable enzyme reaction conditions, nucleic acid substrates, and other reagents necessary for detecting the synthesis reaction products. Further, this kit may supply a reagent in such a manner that the amount of reagent required for a single reaction is dispensed to a reaction vessel.

The kit according to the present invention may comprise a fluorescent probe and a quencher probe instead of a molecular beacon. The kit according to the present invention may also comprise a resolution oligomer of higher-order structure.

The kit comprises information used to adequately perform gene quantitation by single molecule detection according to the present invention. Such information may be attached to the kit as instructions, provided on the package surface, or applied on the package surface as a label.

### EXAMPLES

Hereafter, the present invention is described in greater detail with reference to the examples, although the technical scope of the present invention is not limited thereto.

### Example 1

### Quantitation Using a Target Gene Model

It was confirmed that highly accurate quantitation could be actually carried out by the method of present invention.

(1) Testing Method

A test was performed using a target oligo having part of the hepatitis C virus (HCV) sequence as a target gene model. A padlock probe was allowed to act on a target oligo of known concentration, i.e., 10⁻¹⁶ M, 10⁻¹⁵M, 10⁻¹⁴ M, or 10⁻¹³ M, to perform a ligation reaction. NEB's ligase was employed. A target oligo (SEQ ID NO: 1) at a given concentration and 10 fmol of padlock probe for HCV (SEQ ID NO: 2) were added to a solution containing 10 μl of reaction buffer, the mixture was incubated at 37° C. for 30 minutes to perform a ligation reaction, and the reaction was terminated by deactivating the ligase. Subsequently, NEB's Bst DNA polymerase was used as strand-displacement DNA polymerase, and a primer (SEQ ID NO: 3) and a molecular beacon (SEQ ID NO: 4) were used to perform an RCR reaction. The circularized padlock probe synthesized above, 450 fmol of the primer, and 450 fmol of the molecular beacon for HCV detection, which has been labeled at the 5′ end with FAM and at the 3′ end with BHQ1, were added to a solution containing 15 μl of reaction buffer and substrate dNTP, and the resultant was incubated at 65° C. for 90 minutes to perform an RCR reaction. The obtained sample was analyzed using a detection apparatus as shown in FIG. 5, and the target oligo of interest was quantitated.

| | |
|---|---|
| Target oligo: | |
| 5′-GTA CCA CAA G GCC TTT CGC GAC CCA | (SEQ ID NO: 1) |
| ACA CTA CTC GGC TAG CAG TCT CGC GGG | |
| GGC A-3′ | |
| Padlock probe for HCV: | |
| 5′-TGT TGG GTC GCG AAA GGC CCC TTCT | (SEQ ID NO: 2) |
| CAC TGTT CTC TCAT A GAA GTG ATGA GGG | |
| AACA GAG CAC TCAT CTC TTCT CCC TGTT | |
| AGA CTG CTA GCC GAG TAG-3′ | |
| Primer: | |
| 5′-CTC TGTT CCC TCAT CAC TTCT-3′ | (SEQ ID NO: 3) |
| Molecular beacon for HCV detection: | |
| 5′-(FAM)-CGA CGT CCC TTCT CAC TGTT | (SEQ ID NO: 4) |
| CTC TCAT CAG TCG-(BHQ1)-3′ | |

(2) Results

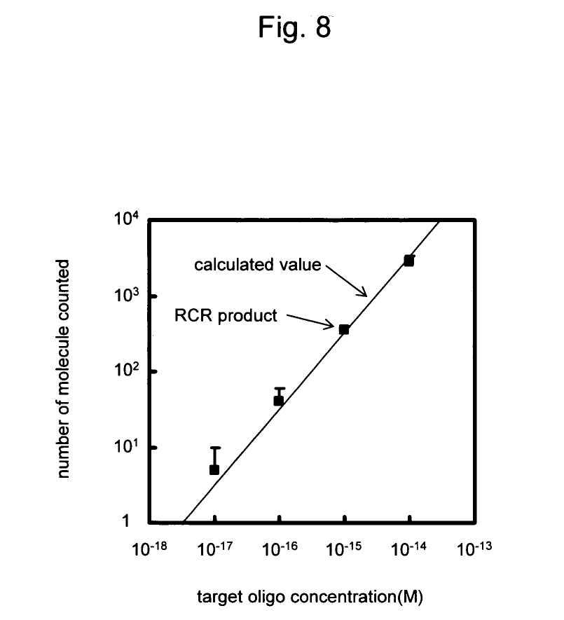

FIG. 8 shows the number of molecules of the RCR product determined by single molecule detection and the number of molecules of the target oligo calculated based on the concentration and the volume subject to detection (6.5×10⁻⁸ l). In a concentration range between 10⁻¹⁷ M and 10⁻¹⁴ M, the number of molecules of the RCR product determined by single molecule detection was found to be consistent with the number of molecules of the target oligo determined. Thus, performance of single molecule detection with lateral incidence via the RCR reaction enabled highly accurate quantitation of the target oligo.

### Example 2

### Quantitative Analysis of Hepatitis C Virus

Hepatitis C viruses at unknown concentration were quantitated by the method of the present invention.

(1) Testing Method

Total RNA extracted from 1 ml of blood was reversely transcribed using a reverse transcription primer (SEQ ID NO: 5) for hepatitis C viruses (HCV), and the resulting cDNA was used as a sample. Reverse transcription was carried out by adding 2 pmol of the following reverse transcription primer, RNA extracted from blood, and Superscript II reverse transcriptase (Invitrogen) to a reaction buffer and incubating the mixture at 42° C. for 50 minutes. Thereafter, ribonuclease H was added to the reaction solution and the resultant was incubated at 37° C. for 20 minutes to decompose RNA remaining in the reaction solution.

Reverse transcription primer for HCV: 5′-TGC TCA TGG TGC ACG GTC TA-3′ (SEQ ID NO: 5)

The ligation reaction was next carried out. NEB's ligase was employed. The first-strand cDNA synthesized above and 10 fmol of padlock probe for HCV (SEQ ID NO: 2) used in Example 1 were added to a solution containing 10 μl of reaction buffer, the mixture was incubated at 37° C. for 30 minutes to perform a ligation reaction, and the ligase was then deactivated. Further, NEB's Bst DNA polymerase was used as strand-displacement DNA polymerase, and a primer (SEQ ID NO: 3) used in Example 1 and a molecular beacon for HCV detection (SEQ ID NO: 4) used in Example 1 were used to perform an RCR reaction. The circularized padlock probe synthesized above, 450 fmol of the primer, and 450 fmol of the molecular beacon for HCV detection, which had been labeled at the 5′ end with FAM and at the 3′ end with BHQ1, were added to a solution containing 15 μl of reaction buffer and substrate dNTP, and the resultant was incubated at 65° C. for 90 minutes to perform an RCR reaction. After the reaction, the obtained sample was analyzed using a detection apparatus as shown in FIG. 5, and the target HCV genes were quantitated.

(2) Results

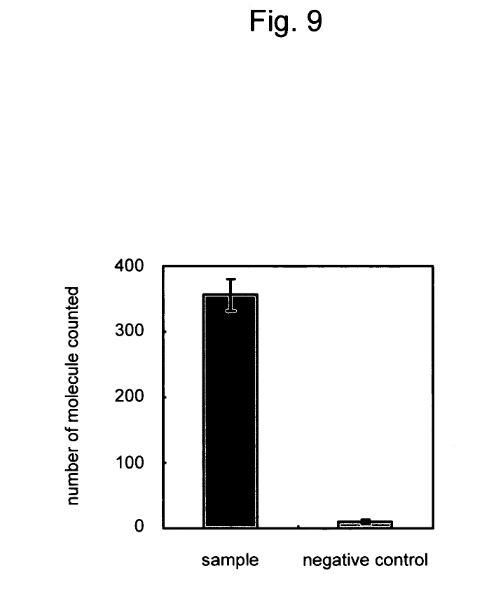

In the same manner as in Example 1, 6.5×10⁻⁷ l of the solution after reaction was analyzed. As a result, the number of molecules was found to be 356, as shown in FIG. 9. This indicates that 5,340 HCV viruses were present in 1 ml of blood before extraction.

### Example 3

### Quantitatively Detection of Hepatitis B Viruses (HBV) Using Fluorescent Probe

In the method according to the present invention, the fluorescent probe and the quencher probe as shown in FIG. 2 were used to quantitate HBV. The quencher probe was designed to have a single base-pair mismatch with the sequence of the fluorescent probe, and this mismatch was found to improve the efficiency of fluorescent labeling.

(1) Testing Method

As a sample, DNA extracted from blood was subjected to hybridization and ligation to the target gene of the padlock probe for HBV (SEQ ID NO: 6). NEB's ligase was employed. The extracted DNA and 10 fmol of padlock probe for HBV (SEQ ID NO: 6) were added to a solution containing 10 μl of reaction buffer, the mixture was incubated at 37° C. for 30 minutes to perform a ligation reaction, and the ligase was then deactivated. Further, NEB's Bst DNA polymerase was used as strand-displacement DNA polymerase, and a primer (SEQ ID NO: 3) used in Example 1 and a fluorescent probe for HBV detection (SEQ ID NO: 7) were used to perform an RCR reaction. The circularized padlock probe synthesized above, 450 fmol of the primer, and 450 fmol of fluorescent probe for HBV detection, which had been labeled at the 5′ end with ROX, were added to a solution containing 15 μl of reaction buffer and substrate dNTP, and the resultant was incubated at 65° C. for 90 minutes to perform an RCR reaction. After the reaction, 600 fmol of quencher probe for HBV was added to quench excess fluorescent probes for HBV detection. The properties of three types of quencher probes for HBV (as shown in SEQ ID NOs: 8, 9, and 10) that hybridize to fluorescent probes for HBV detection were compared. The quencher probe as shown in SEQ ID NO: 8 is composed of the same number of bases as the fluorescent probe, and it completely hybridizes to the fluorescent probe. The quencher probe as shown in SEQ ID NO: 9 is composed of the same number of bases as the fluorescent probe, and it forms a single base-pair mismatch upon hybridization with the fluorescent probe. The quencher probe as shown in SEQ ID NO: 10 is shorter than the fluorescent probe by 3 bases. The obtained samples were analyzed using the detection apparatus shown in FIG. 5 to quantitate hepatitis B viruses.

| Padlock probe for HBV: | |
|---|---|
| 5′-AC YTG TAT TCC CAT CCC A CCC | (SEQ ID NO: 6) |
| TTCT CTC TCAT CAC TGTT AGAA GTG | |
| ATGA GGG AACA GAG CAC TCAT CTC | |
| TTCT CCC TGTT AG AAG GAG AGG AAA | |
| YTG C-3′ | |
| Fluorescent probe for HBV | |
| detection: | |
| 5′-(ROX)-CCC TTCT CTC TCAT CAC | (SEQ ID NO: 7) |
| TGTT-3′ | |
| Quencher probe for HBV: | |
| 5′-AACA GTG ATGA GAG AGAA GGG- | (SEQ ID NO: 8) |
| (BHQ2)-3′ | |
| Quencher probe for HBV: | |
| 5′-AACA GTG ATGA GCG AGAA GGG- | (SEQ ID NO: 9) |
| (BHQ2)-3′ | |
| Quencher probe for HBV: | |
| 5′-A GTG ATGA GAG AGAA GGG-(BHQ2)- | (SEQ ID NO: 10) |
| 3′ | |

(2) Results

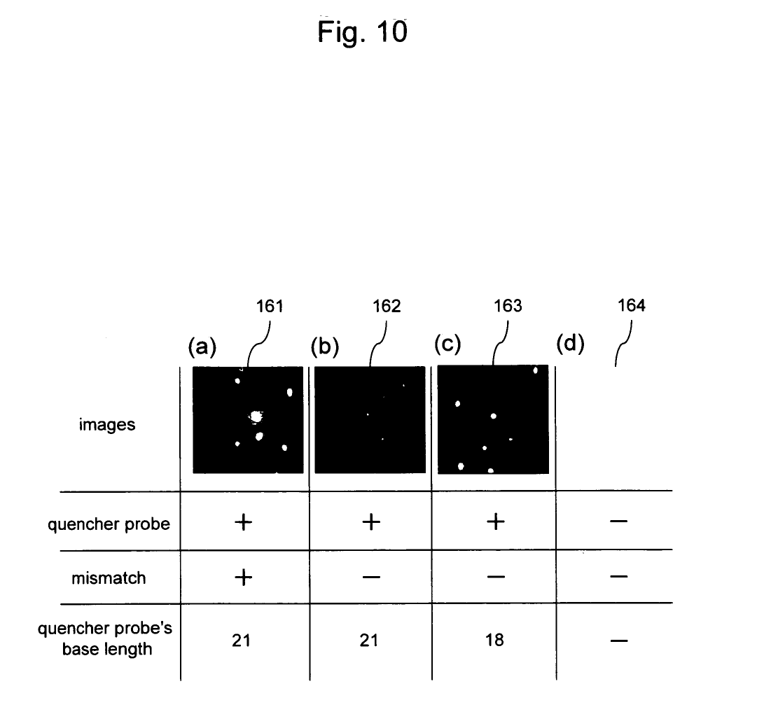

FIG. 10 shows images of ROX-labeled RCR products assayed with the use of various types of quencher probes. These images were obtained with a CCD camera via 10-msec exposures with a 50 μm×50 μm field of view. FIG. 10(a) is an image obtained by detection with the use of a quencher that is designed to have a base-pair mismatch upon hybridization to a fluorescent probe. FIG. 10(b) is an image obtained by detection with the use of a quencher probe that is designed to completely hybridize to a fluorescent probe without forming a base-pair mismatch. FIG. 10(c) is an image obtained by detection with the use of a quencher probe that is designed to have fewer bases than a fluorescent probe. FIG. 10(d) is an image obtained by detection without the addition of a quencher probe. When a quencher probe is not added, the background level is disadvantageously high due to fluorescence derived from excess fluorescent probes in the reaction solution. This produced an image 164, in which a fluorescent-labeled sample could not be distinguished, as shown in FIG. 10(d).

When a quencher probe that is designed to have the same number of bases as the fluorescent probe and to not form a base-pair mismatch is used, thermostability in hybridization between a fluorescent probe and a quencher probe is at the same level as that upon hybridization between an RCR product and a fluorescent probe. This resulted in hybridization of part of the fluorescent probe, which had hybridized to the RCR product, to a quencher probe and the lowered efficiency of labeling with a fluorescent probe. This produced an image 162, in which the fluorescent intensity of the labeled substance was low, as shown in FIG. 10(b).

With the use of a quencher probe that is designed to have the same number of bases as the fluorescent probe and to form a base-pair mismatch (FIG. 10(a)), thermostability in hybridization between a fluorescent probe and a quencher probe is lower than that upon hybridization between an RCR product and a fluorescent probe, as with the case involving the use of a quencher probe that is designed to have fewer bases than a fluorescent probe (FIG. 10(c)). Thus, the fluorescent probe that had hybridized to the RCR product would not hybridize to the quencher probe. This resulted in efficient labeling of the RCR product with the fluorescent probe and production of images 161 and 163 exhibiting sufficiently high fluorescent intensities.

In the method involving the use of the quencher probe into which a mismatch had been introduced, 6.5×10⁻⁷ l of the solution after reaction was assayed in the same manner as in Example 1. As a result, the number of molecules was found to be 2,493 on average. Based on the obtained average, the hepatitis B virus quantity contained in 1 ml of blood before extraction was calculated and the number of hepatitis B viruses was found to be 37,395.

### Example 4

### Quantitative Analysis of Hepatitis C Viruses Using a Fluorescent Probe and a Resolution Oligomer of Higher-Order Structure

According to the method of the present invention, the resolution oligomer of higher-order structure was used to confirm an improvement in efficiency of fluorescent labeling, as shown in FIG. 4.

(1) Testing Method

In the same manner as in Example 2, reverse transcription and hybridization and ligation of a padlock probe were carried out. NEB's Bst DNA polymerase was used as strand-displacement DNA polymerase and the primer used in Example 1 (SEQ ID NO: 3), the fluorescent probe (SEQ ID NO; 11), and the resolution oligomer of higher-order structure (SEQ ID NO: 12) were used to carry out the RCR reaction. The circularized padlock probe synthesized above, 450 fmol of the primer, 450 fmol of the fluorescent probe for HCV detection, which had been labeled at the 5′ end with FAM, and 450 fmol of the resolution oligomer of higher-order structure were added to a solution containing 15 μl of reaction buffer and substrate dNTP, and the resultant was incubated at 65° C. for 90 minutes to perform an RCR reaction. After the reaction, 600 fmol of quencher probe for HCV (SEQ ID NO: 13) was added to quench excess fluorescent probes for HCV detection. The obtained samples were analyzed using the detection apparatus shown in FIG. 5 to quantitate the target hepatitis C viruses.

| Fluorescent probe for HCV | |
|---|---|
| detection: | |
| 5′-(FAM)-CCC TTCT CAC TGTT CTC | (SEQ ID NO: 11) |
| TCAT-3′ | |
| Resolution oligomer of higher- | |
| order structure: | |
| 5′-CAC TCAT CTC TTCT CCC TGTT-3′ | (SEQ ID NO: 12) |
| Quencher probe for HCV: | |
| 5′-A GAG AACA GTG AGAA GGG-(BHQ1)- | (SEQ ID NO: 13) |
| 3′ | |

(2) Results

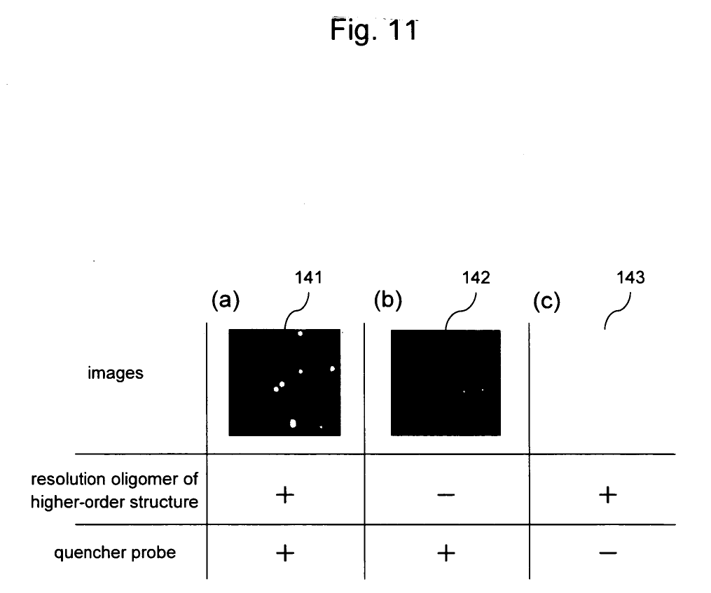

FIG. 11 shows images obtained by assaying the FAM-labeled RCR products. These images were obtained with a CCD camera via 10-msec exposures with a 50 μm×50 μm field of view. FIG. 11(a) is an image of a sample that had been subjected to the reaction with the addition of a resolution oligomer of higher-order structure. FIG. 11(b) is an image of a sample that had been subjected to the reaction without the addition of a resolution oligomer of higher-order structure. FIG. 11(c) is an image of a sample that had been assayed without the addition of a quencher probe. When a quencher probe is not employed, the background level is disadvantageously high due to fluorescence derived from excess fluorescent probes in the reaction solution. This produced an image 143, in which a fluorescent-labeled sample could not be distinguished, as shown in FIG. 11(c). Without the addition of the resolution oligomer of higher-order structure, the efficiency of labeling by a fluorescent probe became low. This produced an image 142, in which the fluorescent intensity of the labeled substance was low, as shown in FIG. 11(b). With the addition of the resolution oligomer of higher-order structure, however, fluorescent probe labeling can be efficiently carried out, as in the case shown in FIG. 11(a). This produced an image 141, in which a single molecule could be detected with sufficiently high fluorescent intensity. Thus, the effects of improving labeling efficiency via the resolution oligomer of higher-order structure were confirmed.

In the method involving the use of the resolution oligomer of higher-order structure, 6.5×10⁻⁷ l of the solution after reaction was assayed in the same manner as in Example 1. As a result, the number of molecules was found to be 127 on average. Based on the obtained average, the number of hepatitis C viruses contained in 1 ml of blood before extraction was determined to be 1,905.

### Example 5

### Simultaneous Assay of a Plurality of Target Genes

A plurality of target genes were simultaneously analyzed by the method of the present invention. Labeling involved the use of two types of molecular beacons.

(1) Testing Method

Blood samples “a” and “b” were sampled from different patients, total RNA extracted therefrom was converted into cDNA using a reverse transcription primer for HCV, and each of the obtained cDNA samples was mixed with DNA extracted from separate blood. Reverse transcription was carried out in the same manner as in Example 2. Subsequently, the following two padlock probes were mixed with the samples and ligation reaction was performed. The padlock probe for HCV used in Example 1 (SEQ ID NO: 2) and the padlock probe for HBV used in Example 3 (SEQ ID NO: 6) were used. The RCR reaction was carried out in the same manner as in Example 2. The primer (SEQ ID NO: 3) that could hybridize to both of the circularized padlock probes was used, and the resolution oligomer of higher-order structure (SEQ ID NO: 12) that could hybridize to both of the RCR products was used. In order to label the RCR products, the molecular beacon for HCV detection (SEQ ID NO: 4) that hybridizes to the RCR product generated from the HCV-derived circularized padlock probe was used in the same manner as in Example 1, and the molecular beacon for HBV detection (SEQ ID NO: 14) that hybridizes to the RCR product generated from the HBV-derived circularized padlock probe was used. The molecular beacon for HBV detection (SEQ ID NO: 14) is ROX-labeled at its 5′ end. The obtained samples were assayed using the detection apparatus as shown in FIG. 7, and the target HCV and HBV genes were quantitated.

| | |
|---|---|
| Molecular beacon for HBV | |
| detection: | |
| 5′-(ROX)-CGA CGT CCC TTCT CTC TCAT | (SEQ ID NO: 14) |
| CAC TGTT ACG TCG-(BHQ2)-3′ | |

(2) Results

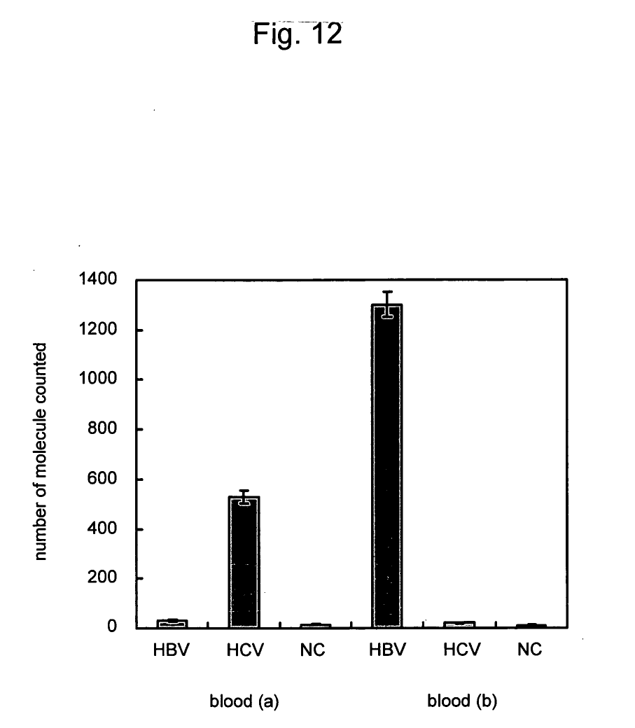

As shown in FIG. 12, hepatitis C viruses were detected in blood sample “a” and hepatitis B viruses were detected in blood sample “b.” The numbers of molecules in 6.5×10⁻⁷ l of viral solutions were 532 and 1,301, respectively. This indicates that 7,980 hepatitis C viruses existed in 1 ml of blood sample “a” and that 19,515 hepatitis B viruses existed in 1 ml of blood sample “b.” The level of the nonexistent viruses detected in the samples was the same as that of the negative control, indicated as “NC” in FIG. 12. Thus, it was confirmed that the method of the present invention allows a plurality of target genes to be simultaneously subjected to single molecule detection.

### INDUSTRIAL APPLICABILITY

The present invention enables highly accurate and highly sensitive quantitation with quantitation limits of 10⁻¹⁸ M. The highly accurate and highly sensitive method of gene quantitation according to the present invention is very useful in the fields of medicine and drug discovery in terms of realizing a reliable diagnosis or confirming therapeutic effects.

Sequence Listing Free Text

SEQ ID NO: 1: description of artificial sequence: target oligo model (part of the HCV sequence)

SEQ ID NO: 2: description of artificial sequence: padlock probe for HCV

SEQ ID NO: 3: description of artificial sequence: primer for producing an RCR product

SEQ ID NO: 4: description of artificial sequence: molecular beacon for HCV detection

SEQ ID NO: 5: description of artificial sequence: reverse transcription primer for HCV

SEQ ID NO: 6: description of artificial sequence: padlock probe for HBV

SEQ ID NO: 7: description of artificial sequence: fluorescent probe for HBV detection

SEQ ID NO: 8: description of artificial sequence: quencher probe for HBV

SEQ ID NO: 9: description of artificial sequence: quencher probe for HBV

SEQ ID NO: 10: description of artificial sequence: quencher probe for HBV

SEQ ID NO: 11: description of artificial sequence: fluorescent probe for HCV

SEQ ID NO: 12: description of artificial sequence: resolution oligomer of a higher-order structure of RCR product

SEQ ID NO: 13: description of artificial sequence: quencher probe for HCV

SEQ ID NO: 14: description of artificial sequence: molecular beacon for HBV detection

1. A method of nucleic acid analysis comprising steps of:
allowing a polynucleotide comprising a first region having a sequence complementary to the target gene (nucleic acid) at the 3′ end, a second region having a sequence complementary to the target gene at the 5′ end, and a third region corresponding to a detection probe to hybridize to the target gene;
allowing the 3′ end of the first region hybridized to the target gene to ligate to the 5′ end of the second region so as to obtain a circularized polynucleotide;
with the use of the circularized polynucleotide as a template, performing a primer extension reaction using a primer having a sequence complementary to part of the circularized polynucleotide and a strand-displacement DNA polymerase;
allowing a detection probe containing a sequence identical to the third region to hybridize to a sequence complementary to the third region that iteratively appears in a single-stranded portion of the extension product; and
optically detecting the amount of the detection probe hybridized to the extension product to thereby quantitate the target gene.

2. The method according to claim 1, wherein the detection probe is labeled with a fluorophore at one of its ends and with a quencher at the other end.

3. The method according to claim 1, wherein the detection probe is labeled with a fluorophore, and a free detection probe is quenched by a quencher probe that is labeled with a quencher and hybridizes specifically to said detection probe.

4. The method according to claim 3, wherein the quencher probe is shorter than the detection probe by 1 to 5 bases or has 1 or 2 base-pair mismatches.

5. The method according to claim 3, wherein the detection probe is allowed to hybridize to the extension product, and the quencher probe is then allowed to hybridize to a free detection probe.

6. The method according to claim 3, wherein the quantity of the quencher probes added is greater than that of the detection probes.

7. The method according to claim 1, wherein an oligomer comprising a sequence identical to an arbitrary fourth region that does not overlap with the third region on the polynucleotide is prepared, the oligomer is allowed to hybridize to the extension product, and the detection probe is then allowed to hybridize to the extension product.

8. The method according to claim 7, wherein the 3′ end of the oligomer, that of the detection probe, and that of the quencher probe are modified to prevent an extension reaction from occurring.

9. A method of simultaneously analyzing two or more types of target genes by the method comprising steps of: allowing the 3′ end of the first region hybridized to the target gene to ligate to the 5′ end of the second region so as to obtain a circularized polynucleotide: with the use of the circularized polynucleotide as a template, performing a primer extension reaction using a primer having a sequence complementary to part of the circularized polynucleotide and a strand-displacement DNA polymerase; allowing a detection probe containing a sequence identical to the third region to hybridize to a sequence complementary to the third-region that iteratively appears in a single-stranded portion of the extension product; and optically detecting the amount of the detection probe hybridized to the extension product to thereby quantitate the target gene;
allowing a polynucleotide comprising a first region having a sequence complementary to the target gene at the 3′ end, a second region having a sequence complementary to the target gene at the 5′ end, and a third region corresponding to a detection probe to hybridize to the target gene;
allowing the 3′ end of the first region hybridized to the target gene to ligate to the 5′ end of the second region so as to obtain a circularized polynucleotide;
with the use of the circularized polynucleotide as a template, performing a primer extension reaction using a primer having a sequence complementary to part of the circularized polynucleotide and a strand-displacement DNA polymerase;
allowing a detection probe containing a sequence identical to the third region to hybridize to a sequence complementary to the third region that iteratively appears in a single-stranded portion of the extension product; and
optically detecting the amount of the detection probe hybridized to the extension product to thereby quantitate the two or more types of target genes.

10. A kit for nucleic acid analysis comprising the following (1) to (4):
(1) a 60- to 200-bp polynucleotide comprising a first region having a sequence complementary to the target gene at the 3′ end, a second region having a sequence complementary to the target gene at the 5′ end, and an arbitrary third region;
(2) a ligase;
(3) a strand-displacement DNA polymerase; and
(4) a 15- to 30-bp detection probe comprising a sequence identical to the third region and labeled with a fluorophore at one of its ends and with a quencher at the other end.

11. The kit for nucleic acid analysis according to claim 10, which comprises a 15- to 30-bp detection probe comprising a sequence identical to the third region and labeled with fluorophores at its ends and a 15- to 30-bp quencher probe hybridizing specifically to the detection probe and labeled with quenchers, instead of the detection probe (4).

12. The kit for nucleic acid analysis according to claim 10, wherein the quencher probe is shorter than the detection probe by 1 to 5 bases or has 1 or 2 base-pair mismatches.

13. The kit for nucleic acid analysis according to claim 10, which further comprises a 15- to 30-bp oligonucleotide having a sequence identical to the fourth region that does not overlap with the third region on the polynucleotide.
